(19) United States
(12) Patent Application Publication
(10) Pub. No.: US 2005/0070983 A1
(43) Pub. Date: Mar. 31, 2005

(54) Lead system having lead body with minimized cross-section

(76) Inventors: Jaime L. Rugnetta, White Bear Lake, MN (US); Mary S. Wentorf, Roseville, MN (US); Gregory R. Ley, Blaine, MN (US); Christopher M. Zerby, New Brighton, MN (US); Lyle A. Bye, Lino Lakes, MN (US); Paul E. Zarembo, Vadnais Heights, MN (US); Brian D. Soltis, St. Paul, MN (US); Jeffrey T. Bartig, Maplewood, MN (US)

Correspondence Address: Schwegman, Lundberg, Woessner & Kluth, P.A., P.O. Box 2938, Minneapolis, MN, 55402, US

(21) Appl. No.: 10/670,985

(22) Filed: Sep. 25, 2003

(51) Int. Cl. A61N 1/05

(52) U.S. Cl. 607/122

(57) ABSTRACT

A medical device includes one or more tines having a first position and a second position. The tines are coupled with a body including one or more recessed portions thereon. In one of the first or second positions, the one or more tines are disposed adjacent one of the recessed portions of the body.

### TECHNICAL FIELD

The present invention relates generally to medical devices. More particularly, it pertains to insertable medical instruments having a minimized cross-section.

### BACKGROUND OF THE INVENTION

Electrodes have been implanted in the body for electrical cardioversion or pacing of the heart. More specifically, electrodes implanted in or about the heart have been used to reverse certain life threatening arrhythmias, or to stimulate contraction of the heart, where electrical energy is applied to the heart via the electrodes to return the heart to normal rhythm.

The electrodes are often fixated within passages or within chambers of the heart through use of one or more tines which extend from the lead body. While the tines are effective in fixating the electrode or lead body, there are drawbacks to their use. For example, the extension of the tine away from the body results in an increased diameter of the lead body. This limits the size of the introducer that can be used, as well as the location at which the lead can be implanted.

Accordingly, what is needed is a medical device for fixating within a body that overcomes the above drawbacks.

### SUMMARY OF THE INVENTION

A medical device such as a lead assembly includes a lead body with a tine interface section. The lead further includes at least one tine that is coupled with the lead body at a tine coupling portion. The tine includes a first position extended away from the lead body, and a second collapsed position. Along the tine interface section is at least one first recessed portion and a second recessed portion. In one option, the first recessed portion is recessed away from the bottom surface of the tine when the tine is disposed in the second collapsed position. In another option, the first recessed portion has a different cross-sectional shape than the second recessed portion.

Several additional options for the lead assembly are as follows. For example, in one option, the at least one first recessed portion extends only a portion around a perimeter of the lead body. In another option, the first recessed portion has a smaller cross-sectional area than the second recessed portion, for example, about 10 percent smaller.

A method includes disposing a conductor within a lead body where the lead body includes a tine interface portion. The method further includes coupling one or more tines with the lead body, and the one or more tines are collapsible from a first extended position to a second collapsed position. The method also includes forming a first recessed portion and a second recessed portion, and forming the first recessed portion includes recessing the first recessed portion away from a bottom surface of the tine when the one or more tines are disposed in the second collapsed position.

These and other embodiments, aspects, advantages, and features will be set forth in part in the description which follows, and in part will become apparent to those skilled in the art by reference to the following description and referenced drawings or by practice of the invention. The aspects, advantages, and features of the invention are realized and attained by means of the instrumentalities, procedures, and combinations particularly pointed out in the appended claims.

### BRIEF DESCRIPTION OF THE DRAWINGS

FIG. 1 is a view illustrating a lead implanted within a heart constructed in accordance with one embodiment.

FIG. 2A is a side cross-sectional view illustrating a portion of a lead constructed in accordance with one embodiment.

FIG. 2B is a cross-sectional view taken along 2B-2B of FIG. 2A.

FIG. 3 is a side elevational view illustrating a portion of a lead constructed in accordance with one embodiment.

FIG. 4 is a cross-sectional view taken along 4-4 of FIG. 3.

FIG. 5 is a cross-sectional view taken along 5-5 of FIG. 3.

FIG. 6 is a side elevational view illustrating a portion of a lead assembly constructed in accordance with one embodiment.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

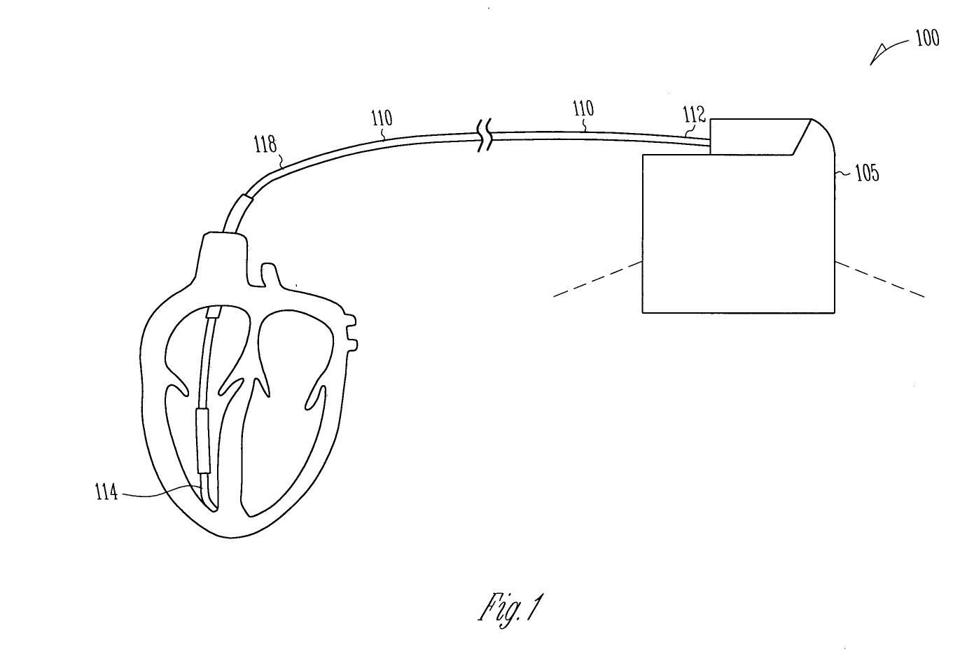

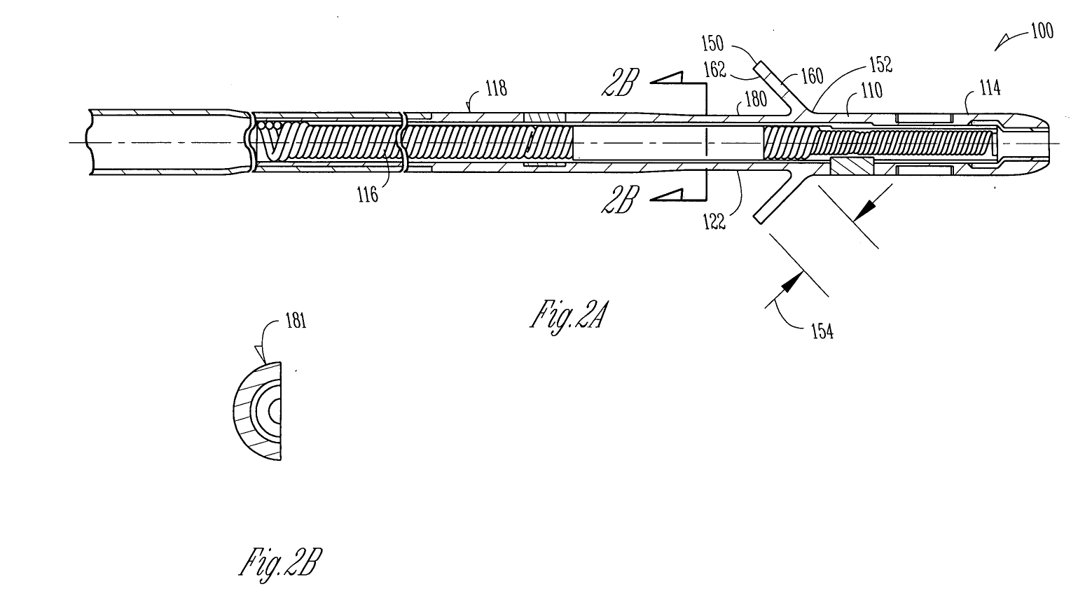

FIG. 1 illustrates a lead assembly 100 constructed in accordance with one embodiment. The lead assembly includes a lead body 110, such as an elongate lead body extending from a lead proximal end 112 to a lead distal end 114, with an intermediate portion therebetween 118. The lead body, in one option, has a substantially circular cross-section. Disposed within the lead body 110 is at least one conductor 116 (FIG. 2A). The lead body includes a tine interface portion 122 (FIG. 2A), as discussed further below. In one option the lead assembly 100 is electrically coupled with an electronics unit, such as a pulse generator 105. The lead assembly 100 further includes one or more tines 150, an example shown in greater detail in FIGS. 2A and 2B.

FIG. 2A illustrates a cross-sectional view of one embodiment of a lead assembly 100. Disposed, in one option, near or at a distal end 114 of the lead body 110 are the one or more tines 150. The one or more tines 150 are coupled with the lead body 110 at a tine coupling portion 152, and the one or more tines are defined in part by a tine length 154. The one or more tines 150 extend away from the lead body 110 in a first position, as illustrated in FIG. 2A. The one or more tines 150 further include a second collapsed position, as illustrated in FIG. 6. The lead body 110, as discussed above, includes a tine interface portion 122 that, in one option, extends for approximately the same length as the tine length 154.

In another option, the tine interface portion 122 is slightly longer than the tine length 154, allowing for elongation of the tines 150. For example, the one or more tines 150 can be formed of material that will elongate or stretch. In one option, the outer diameter of the tines and lead body can be slightly larger than the device through which they are being inserted, such as a catheter. The friction between the inner surface of the device such as the catheter causes the tines to stretch and the tines are stretched and are elongated, allowing an overall lower profile of the outer diameter of the tines and lead body.

The one or more tines 150 include a top surface 160 and a bottom surface 162. In one option, the top surface 160 of the one or more tines includes a slightly rounded surface. In another option, the top surface 160 includes two surfaces that are slightly angled relative to the bottom surface of the one or more tines 150. The bottom surface 162, in one option, is substantially planar, or optionally substantially flat, such that the bottom surface 162 can lie flat against another planar surface, such as a planar recessed portion as discussed further below.

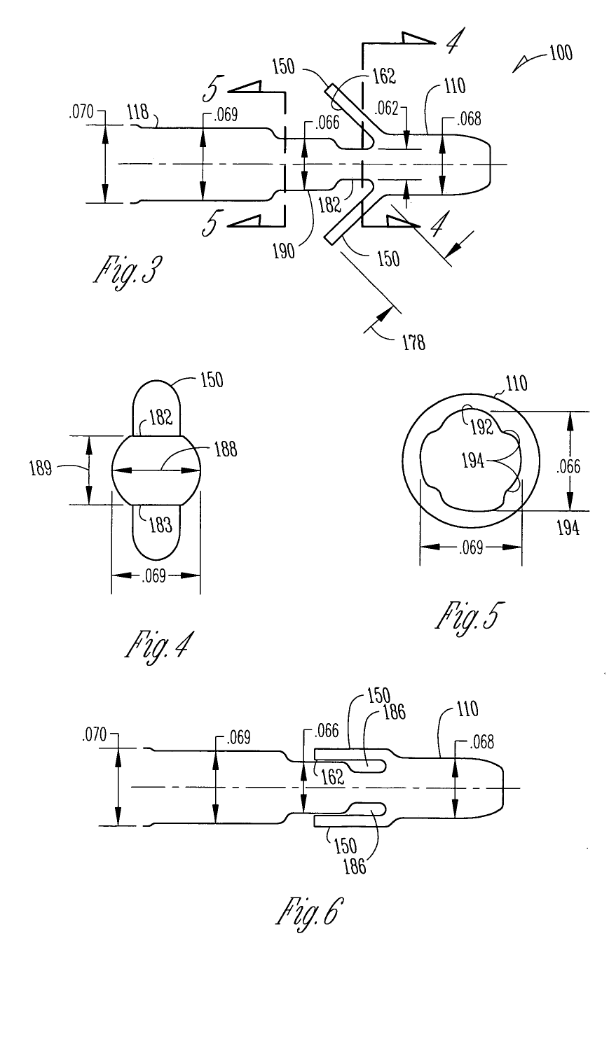

The lead body 110, in one option, includes at least one recessed portion 180, where a portion of the lead body 110 is recessed away in diameter away from an outer diameter defined at the intermediate portion 118. In another option, the lead body includes two opposed recessed portions 181, as illustrated in FIGS. 2A and 2B. In yet another option, the lead body includes two or more recessed portions disposed at different longitudinal locations, for example, as illustrated in FIGS. 3-5.

FIG. 3 illustrates a side elevational view of a lead assembly 100 in one embodiment. The lead body includes a first recessed portion 182 that has a first cross-sectional shape, and a first cross-sectional area, as illustrated in FIG. 4. The lead body 110 further includes a second recessed portion 190 that has a second cross-sectional shape, and a second cross-sectional area, as illustrated in FIG. 5. In one option, the first cross-sectional area is smaller than the second cross-sectional shape, for example, the first cross-sectional area is about 10% smaller than the second cross-sectional area. In another option, the first cross-sectional shape is different than the second cross-sectional shape.

The first recessed portion 182, in one option, is recessed to facilitate the collapsibility of the one or more tines, as the recessed portion allows for the movement of material under the tine coupling portion to move as the one or more tines are collapsed. For example, in the second collapsed position (FIG. 6), the first recessed portion 182 allows for a void or space 186 between the first recessed portion 182 and the bottom surface of the one or more tines 150, allowing for example, for movement of material coupling the tine to the lead body 110 as the tine is collapsed. In one option, the first recessed portion 182 is less than the overall tine length 178. In another option, the first recessed portion 182 does not extend fully around the perimeter of the lead body 110, as illustrated for example in FIG. 4. For example, in one option, the first recessed portion 182 is a flat 183 that only extends partially around the perimeter of the lead body 110, or the lead body 110 is non-circular. In another option, the first recessed portion 182 includes multiple recessed portions. In yet another option, the first recessed portion 182 includes a first transverse dimension 188 and a second transverse dimension 189. Optionally, the first transverse dimension 188 is greater than the second transverse dimension 189.

The second recessed portion 190, in one option, is recessed to facilitate the collapsibility of the one or more tines 150, as the second recessed portion 190 allows for the bottom surface 162 of the tine 150 to be disposed adjacent thereto when the tine 150 is disposed in the second position (FIG. 6). For example, in the second collapsed position (FIG. 6), the bottom surface 162 of the tine 150 is disposed directly adjacent to the second recessed portion 190, for example, contact the surface 192 of the second recessed portion 190. In one option, the second recessed portion 190 does not extend fully around the perimeter of the lead body 110. For example, in one option, the second recessed portion is segmented into portions that only extend partially around the perimeter of the lead body 110, or the lead body 110 is non-circular. In another option, the second recessed portion 190 includes multiple recessed portions 194.

The tines also provide a mechanism which is also adapted for anchoring the medical device against inadvertent removal from the patient, and/or provides for compressibility of the tines which assists in deployment of the medical device through the patient. The one or more tines, in one embodiment, are formed from a flexible material, such as silicone, polyurethane, or fluropolymers (e.g. PTFE, ETFE, FEP, PFA). Alternatively, the one or more tines are formed from other materials, such as a bioresorbable material.

A method includes disposing a conductor within a lead body where the lead body includes a tine interface portion. The method further includes coupling one or more tines with the lead body, and the one or more tines are collapsible from a first extended position to a second collapsed position. The method also includes forming a first recessed portion and a second recessed portion, and forming the first recessed portion includes recessing the first recessed portion away from a bottom surface of the tine when the one or more tines are disposed in the second collapsed position.

In one option, the method further includes collapsing the one or more tines to the second collapsed position at a tine interface portion. In another option, forming the first recessed portion includes forming a non-circular cross-section at the tine interface portion, and optionally forming a second non-circular cross-section at the tine interface portion. In yet another option, forming the first recessed portion includes forming the first recessed portion with a smaller cross-sectional area than the second recessed portion. In one option, forming the first and second recessed portions includes forming the first recessed portion with at least one of a different cross-section than the second recessed portion or a different cross-sectional shape than the second recessed portion. The method can further include incorporating the various recessed portions, and combinations thereof as further discussed above, and/or illustrated in the Figures.

Advantageously, the tines and/or the body of the medical device can collapse to a low profile when passing through restricting diameters such as introducers or vasculature, thus creating minimal drag during placement of the medical device. When unrestricted by the restricting diameters, the construction of the tines of the medical device are adapted to offer rigidity that will cause the one or more tines to stand out away from the medical device body, thereby creating a mechanism for anchoring the medical device. One example of a medical device which is useful with the one or more tines is a lead placement. When the tines are used in combination with the lead, the lead can be anchored within a heart chamber, artery, or vein using the tines as a passive fixation until tissue end-growth occurs. Alternatively, the lead can be introduced intravenously as the tines collapse to a low profile.

Lead deployment is improved since compressibility of the medical device is improved and the amount of drag is reduced as the lead is displaced through a vein. Furthermore, the manner in which the lead body is made allows for the leads to be made smaller, which reduces complications and trauma to the patient. This also allows for increased flexibility in lead placement, as smaller leads can pass through smaller passages, resulting in a wider variation of therapies that can be applied.

It is to be understood that the above description is intended to be illustrative, and not restrictive. Many other embodiments will be apparent to those of skill in the art upon reviewing the above description. For instance, the one or more tines can be used with a wide variety of medical devices. The scope of the invention should, therefore, be determined with reference to the appended claims, along with the full scope of equivalents to which such claims are entitled.

1. A lead assembly comprising:
a lead body extending from a lead proximal end to a lead distal end and having an intermediate portion therebetween, the lead body including a tine interface section;
at least one tine coupled with the lead body at a tine coupling portion, each at least one tine having a top surface and a bottom surface, the at least one tine having a first position extended away from the lead body, the at least one tine having a second collapsed position;
at least one first recessed portion formed on the lead body at a first longitudinal location along the tine interface section of the lead body;
at least one second recessed portion formed on the lead body at a second longitudinal location along the section of the lead body;
the at least one first recessed portion longitudinally disposed between the tine coupling portion and the at least one second recessed portion; and
wherein the at least one first recessed portion is recessed away from the bottom surface of the at least one tine when the at least one tine is disposed in the second collapsed position.

2. The lead assembly as recited in claim 1, wherein the at least one first recessed portion extends only a portion around a perimeter of the lead body.

3. The lead assembly as recited in claim 1, wherein the lead body has a first cross-sectional area at the at least one first recessed portion, and the lead body has a second cross-sectional area at the at least one second recessed portion, and the first cross-sectional area is smaller than the second cross-sectional area.

4. The lead assembly as recited in claim 1, wherein the lead body has a first cross-sectional area at the tine interface portion, and the lead body has a second cross-sectional area at a second area between the at least one tine and the lead distal end, and the first cross-sectional area is less than 10% smaller than the second cross-sectional area.

5. The lead assembly as recited in claim 1, wherein the lead body has a first transverse dimension and a second transverse dimension each at a longitudinal location along the first recessed portion, and the first transverse dimension is greater than the second transverse dimension.

6. The lead assembly as recited in claim 1, wherein the at least one tine is defined in part by a tine length, and the at least one first recessed portion has a second longitudinal length, and the second longitudinal length is less than the tine length.

7. The lead assembly as recited in claim 1, wherein the intermediate portion of the lead body has a first cross-section, the first recessed portion has a second cross-section, and the second recessed portion has a third cross-section, and the first cross-section, the second cross-section, and the third cross-section are each different from one another.

8. A lead assembly comprising:
a lead body extending from a lead proximal end to a lead distal end and having an intermediate portion therebetween, the lead body having a tine interface section;
at least one tine coupled with the lead body at a tine coupling portion, each at least one tine having a top surface and a bottom surface, the at least one tine having a first position extended away from the lead body, the at least one tine having a second collapsed position;
at least one first portion formed on the lead body at a first location along the tine interface section of the lead body, the at least one first portion having a first cross-sectional shape;
at least one second portion formed on the lead body at a second location along the tine interface section of the lead body, the at least one second portion having a second cross-sectional shape;
the first cross-sectional shape is different that the second cross-sectional shape; and
the at least one first recessed portion longitudinally disposed between the tine coupling portion and the at least one second recessed portion.

9. The lead assembly as recited in claim 8, wherein the first cross-sectional shape has a substantially smaller cross-sectional area than the second cross-sectional shape.

10. The lead assembly as recited in claim 8, wherein the first cross-sectional shape includes one or more recessed portions.

11. The lead assembly as recited in claim 10, wherein the one or more recessed portions extend only a portion around a perimeter of the lead body.

12. The lead assembly as recited in claim 8, wherein the second cross-sectional shape includes one or more recessed portions.

13. The lead assembly as recited in claim 10, wherein the one or more recessed portions extend only a portion around a perimeter of the lead body.

14. The lead assembly as recited in claim 8, wherein the at least one first portion is recessed away from the bottom surface when the at least one tine is disposed in the collapsed position.

15. A method comprising:
disposing a conductor within a lead body, the lead body including a tine interface portion;
coupling one or more tines with the lead body, the one or more tines collapsible from a first extended position to a second collapsed position, the one or more tines having a top surface and a bottom surface;
forming a first recessed portion along the lead body at a first longitudinal location along the tine interface portion, wherein forming the first recessed portion includes recessing the first recessed portion away from the bottom surface when the one or more tines are disposed in the second collapsed position; and
forming a second recessed portion along the lead body at a second longitudinal location along the tine interface portion.

16. The method as recited in claim 15, further comprising collapsing the one or more tines to the second collapsed position at a tine interface portion.

17. The method as recited in claim 16, wherein forming the first recessed portion includes forming a non-circular cross-section at the tine interface portion.

18. The method as recited in claim 17, wherein forming the second recessed portion includes forming a second non-circular cross-section at the tine interface portion.

19. The method as recited in claim 15, wherein forming the first recessed portion includes forming the first recessed portion with a smaller cross-sectional area than the second recessed portion.

20. The method as recited in claim 15, wherein forming the first and second recessed portions includes forming the first recessed portion with at least one of a different cross-section than the second recessed portion or a different cross-sectional shape than the second recessed portion.
